(19) United States
(12) Patent Application Publication
(10) Pub. No.: US 2005/0033227 A1
(43) Pub. Date: Feb. 10, 2005

(54) Sclerosing catheter and method for sclerosing blood vessels, in particular veins

(76) Inventor: Jens P. Brodersen, Hannover (DE)

Correspondence Address: SHLESINGER, ARKWRIGHT & GARVEY LLP, 3000 South Eads Street, Arlington, VA, 22202, US

(21) Appl. No.: 10/936,674

(22) Filed: Sep. 9, 2004

Related U.S. Application Data
(63) Continuation of application No. PCT/EP03/02063, filed on Feb. 28, 2003.

(30) Foreign Application Priority Data
Mar. 9, 2002 (DE) 202 03 840.8
Jul. 25, 2002 (DE) 102 33 794.2

(51) Int. Cl. A61M 29/00

(52) U.S. Cl. 604/102.01

(57) ABSTRACT

Catheter for sclerosing or sclerotherapy of blood vessels, especially veins, may include at least two lumina. In order to stop blood flow, a first lumen may be provided with a balloon-shaped element which may be filled with air, for example, and inflated. For applying a sclerosing agent, a second lumen including at least one outlet which may be stationary relative to the balloon-shaped element may be provided.

### CROSS-REFERENCE TO RELATED APPLICATIONS

This application is a continuation of U.S. application no. PCT/EP03/02063, filed Feb. 28, 2003, which claims the priority of German application no. 102 33 794.2, filed Jul. 25, 2002, and which also claims the priority of German application no. 202 03 840.8, filed Mar. 9, 2002, and each of which is incorporated herein by reference.

### FIELD OF THE INVENTION

The invention concerns a sclerotherapy or sclerosing catheter for sclerotherapy or sclerosing of blood vessels, particularly veins.

### BACKGROUND OF THE INVENTION

In DE 199 57 168 AI, a catheter for treatment of leg vein thromboses is described which has three lumina or cavities, of which a first cavity has a first balloon-like element which can be inflated and filled with air. The catheter consists of a flexible material and has a second cavity which serves to carry a lyticum used to dissolve the blood clots occurring in a thrombosis. For the outflow of the lyticum, there is an outlet opening in the second cavity.

DE 693 26 572 T2 and EP 0 557 80 B1 describe a hemodialysis catheter which has multiple cavities.

DE 691 10 467 T2 and EP 0 533 816 B1 describe a catheter for the administration of medications which has one cavity which is connected to a balloon-like element which can be filled with air and inflated.

The administration of the medication is performed using the balloon-like element, which has outlet openings for the medication for this purpose. In order to allow the catheter to reach difficult to access locations in the human body, it is flexible in construction.

DE 698 22 456 T2 and EP 0 364 799 B1 describe a catheter for chemotherapy which has a first cavity connected to balloon-like elements which can be filled with air and inflated. The catheter also has a second cavity to carry a chemotherapeutic agent; the second cavity has multiple outlet openings for the chemotherapeutic agent. The outlet openings are arranged between two balloon-like elements separated from one another along the axial direction of the catheter. Outside the area between the balloon-like elements in which the outlet openings are located, the catheter is flexible in construction, in order to permit the catheter to reach even difficult to access locations in the human body.

WO 01/2861 A2 describes a thrombectomy catheter with multiple cavities, of which one has a balloon-like element which can be filled with air and inflated.

FR 2 803 532 describes an arteriotomy catheter which has two balloon-like elements separated from one another along an axial direction which can be filled with air and inflated. In order to allow the catheter to reach difficult to access locations in the human body, the catheter is flexible in construction.

WO 02/05887 A2 describes a catheter for the administration of medication which has a first cavity connected to two balloon-like elements which can be filled with air and inflated. The catheter has a second cavity which serves to administer the medication and has outlet openings which are located along an axial direction of the catheter between the balloon-like elements. In order to allow the catheter to reach difficult locations in the human body, the catheter is flexible in construction.

US Patent Publication No. 2002/0010418 A1 discloses a multicavitied sclerotherapy catheter of the described type, with multiple tubes, which has an inflatable balloon at the end of one tube. The other tubes have a cavity which serves to carry air to the balloon in order to inflate it and plug the blood flow in the vein. The sclerotherapy catheter also has a self-inflating second balloon which can slide relative to the first balloon, and is connected with a ring-shaped space between the first tube and an additional tube, in which the ring-shaped space serves to carry a medication to the second balloon, which has outlet openings for the medication. The sclerotherapy catheter is first introduced into a vein, after which the balloon is inflated as soon as the desired position is reached. Finally, an external tube part of the sclerotherapy catheter is retracted, so that the self-inflating second balloon inflates. Then a medication is introduced into the self-inflating balloon through the ring-shaped space between the two tubes, which escapes through the outlet openings in the balloon, thus being applied to the inner wall of the blood vessel being treated. Finally, the balloon is pulled back in the lengthwise direction of the vein, so that the medication is distributed along the inner wall of the blood vessel.

A disadvantage of this design is the relatively complicated construction of the sclerotherapy catheter, particularly with respect to the second, movable balloon, which must be pushed along inside the varicose vein in the axial direction in order to distribute the appropriate medication evenly along the inner wall of the vein. This makes the well-known sclerotherapy catheter expensive to manufacture.

An object of the present invention is thus to avoid the aforementioned disadvantages, and in particular to make a sclerotherapy or sclerosing catheter available which is simple in construction, yet still enables an even application of the sclerosant to inner wall of a blood vessel.

This object is achieved by the inventive sclerotherapy catheter in accordance with the invention.

Sclerotherapy catheter for sclerotherapy of blood vessels, particularly veins, in accordance with the invention may include at least two cavities. A first cavity may have a balloon-like element which can be filled with air and inflated to block the blood vessel, and a second cavity may have at least one outlet opening fixed in location relative to the first balloon-like element for the application of a sclerosant or sclerosing agent. The second cavity may be constructed substantially rigid along its entire length, so that upon generation of an underpressure required for the evacuation of excess sclerosant from the blood vessel into the second cavity, the closure of the second cavity by elastic deformation of the interior walls of the second cavity is prevented.

The inventive sclerotherapy catheter for sclerotherapy of blood vessels, particularly veins may have at least two lumina or cavities. To plug the blood flow, the first cavity has a first balloon-like element which can be filled with air and inflated. The second cavity has an outlet opening for application of the sclerosant that is fixed in position relative to the first balloon-like element.

In contrast to the state of the art described above, no element movable in the axial direction, provided with outlet openings for application to a vessel inner wall, is necessary, since it has proved surprisingly that at least one fixed outlet opening is fully sufficient, because the partial or complete spasm of the blood vessel triggered by the sclerosant causes a peristalsis-like restriction due to the reduction in diameter of the blood vessel, leading to the simultaneous movement of the sclerosant to parts of the vessel inner wall which are not yet networked.

Due to this surprising invention, such a simple structural form according to the invention is possible, as has been achieved in accordance with the invention.

The sclerotherapy catheter itself may be constructed as a plastic tube, of polyurethane for example, while the outlet openings can be slits or holes. Generally the length of the sclerotherapy catheter is between 30 and 100 cm, with an overall outer diameter of between 2 and 5 mm. The first balloon-like element which can be filled with air and inflated is generally made of polyurethane, silicon, or rubber. In the inflated state, the diameter of the balloon-like element is generally 2 to 4 cm. Generally, at least one outlet opening for application of the sclerosant is several millimeters distant from the balloon-like element.

According to the invention, the second cavity is constructed sufficiently rigidly along most of its overall length such that when an underpressure is generated in the second cavity to evacuate excess sclerosant from the blood vessel, the closure of the second cavity due to contact of the second cavity's inner walls is prevented. The evacuation of excess sclerosant after conclusion of the sclerotherapy of the treated blood vessel is desirable in order to prevent the undesired remaining of the sclerosant in the patient's body after treatment, as such might possibly lead to health hazards there. In order to evacuate the sclerosant, an underpressure is generated in the second cavity, through which the sclerosant, possibly mixed with blood, is evacuated from the treated blood vessel, and sucked in through the outlet openings into the second cavity of the catheter. In traditional catheters made from particularly flexible plastic tubing, the generation of an underpressure for evacuation of the sclerosant would lead to the elastic deformation of the walls of the second cavity, until the opposing areas of the inner wall of the second cavity came into contact and thus closed the second cavity off, so that the evacuation of the sclerosant from the blood vessel would be prevented. To prevent such a closure of the second cavity by elastic deformation of the walls of the second cavity, the rigidity of the second cavity is selected in the invention such that a closure of the second cavity upon generation of the underpressure required for the evacuation of excess sclerosant from the blood vessel is prevented.

To enable the introduction of the catheter into the blood vessel even if the blood vessel is bent, the inventive catheter can be flexible in construction. It is important that the rigidity of the second cavity is great enough to prevent closure of the second cavity upon generation of the underpressure required for evacuation of excess sclerosant into the second cavity.

Particularly in the case of so-called perforating veins, that is, connecting veins between the surface or superficial and the deep vein systems, it is particularly advantageous if the first cavity has a second balloon-like element capable of being filled with air and inflated for the further closure of the blood vessel offset relative to the outlet opening, where the outlet opening is located between the first and second balloon-like elements in order to prevent the escape of the sclerosant through the perforating veins. Since both balloon-like elements are parts of a single cavity, they inflate nearly synchronously.

A further advantageous embodiment of the invention results when a third cavity has a second balloon-like element offset relative to the outlet opening that can be filled with air and inflated for the further closure of the blood vessel, where the outlet opening is located between the first and second balloon-like elements, since in this way an independent filling and inflation with air is possible for both balloon-like elements.

So that the overpressure present in the balloon-like elements can be comfortably retained in the inflated state without continually needing to add air, it is advantageous when the first and/or third cavity has an air stopping system, in particular a valve, rubber stoppers, or a compression clamp, in order to block air from escaping out of the cavity.

Generally the catheter ends serving to carry air and medication have differently colored markings in order to avoid confusion.

An extraordinarily advantageous further embodiment of the invention specifies that the distal end of the sclerotherapy catheter is bent or angled at an angle of less than 45°, preferably of about 30°, from the lengthwise axis of the sclerotherapy catheter. In this embodiment, the introduction of the sclerotherapy catheter into curved blood vessels is also simplified. If the distal end of the sclerotherapy catheter encounters an obstruction during introduction into the blood vessel, then the sclerotherapy catheter can be moved into an orientation by rotation around the lengthwise axis in which the distal end of the sclerotherapy catheter follows the path of the blood vessel.

Basically, the walls of the first cavity and/or the second cavity and/or the third cavity can be constructed in a largely elastically rigid form. To simplify the introduction of the sclerotherapy catheter into the blood vessel, it is practical if the walls of the first cavity and/or the second cavity and/or the third cavity consist at least in part of an elastically flexible material. In this way, the catheter can be elastically deformed during introduction into the blood vessel, although the second cavity is sufficiently rigid along its entire length that upon generation of the underpressure required to evacuate excess sclerosant from the blood vessel, a closure of the second cavity by elastic deformation of the inner walls of the second cavity is prevented.

The required rigidity of the second cavity can basically be achieved in that the walls of the second cavity are constructed of a sufficiently rigid material. An advantageous further development of the invention specifies stabilization structures to rigidify the second cavity. In this embodiment of the invention, the walls of the second cavity can in principle consist of a highly elastic material. The rigidity of the second cavity to prevent closure of the second cavity upon generation of an underpressure is in this embodiment achieved using the stabilization structures.

In the aforementioned embodiment, the stabilization structures can have a rib extending in the radial direction between the inner walls of the second cavity, or least one bar extending in the radial direction between the inner walls of the second cavity, as specified in one further development. A further development of the aforementioned embodiment specifies that the rib or the ribs, or the bar or bars, extend in the axial direction of the second cavity largely over the entire length of the second cavity. In this embodiment, the same rigidity is attained over the entire length of the second cavity.

An additional further embodiment of the invention specifies that multiple ribs or bars are provided which each extend in the axial direction over a short second of the length of the second cavity and are spaced out from one another in the axial direction. In this embodiment, a high rigidity can be attained in the axial locations where the ribs or bars are located, while the catheter can be constructed flexibly in the areas between the ribs or bars in the axial direction. In this way, it is on the one hand prevented that the second cavity closes upon generation of an underpressure due to the contact of opposing areas of its inner walls. On the other hand, due to the flexibility introduced in the areas between the ribs or bars, the insertion of the catheter into the blood vessel is eased.

In accordance with various requirements, the second cavity can be subdivided into at least two individual cavities by the rib or ribs, or bar or bars, as one further development specifies.

In the aforementioned embodiment, the individual cavities can be separated from one another or communicate with one another, as other further embodiments specify.

Using the following embodiments of the invention as examples, the invention will now be explained in more detail.

Relative terms such as up, down, left, and right, are for convenience only and are not intended to be limiting.

### BRIEF DESCRIPTION OF THE DRAWINGS

FIG. 1 shows a schematic representation of a first embodiment of the inventive sclerotherapy catheter;

FIG. 2 shows a schematic representation of a second embodiment of the inventive sclerotherapy catheter;

FIG. 3 shows a schematic representation of a third embodiment of the inventive sclerotherapy catheter;

FIG. 4 shows a schematic representation of a fourth embodiment of the inventive sclerotherapy catheter;

FIG. 5 shows a cross-section of a fifth embodiment of the inventive sclerotherapy catheter;

FIG. 6 shows a cross-section of a sixth embodiment of the inventive sclerotherapy catheter;

FIG. 7 shows a cross-section of a seventh embodiment of the inventive sclerotherapy catheter; and

FIG. 8 shows a schematic representation of an axial section through a sclerotherapy catheter as in FIG. 4.

In the Figures, identical or corresponding components are labeled with the same labels.

### DETAILED DESCRIPTION OF THE INVENTION

In FIG. 1, a first embodiment of the inventive sclerotherapy or sclerosing catheter is shown schematically. The sclerotherapy catheter has a first tube-formed or tube-shaped cavity 1, which is connected on one end to a first balloon-like element 3 over an inner opening. The sclerotherapy catheter opens on its other end into a tube-formed or tube-like opening 7 over which the air to be applied can be fed in using a syringe-like system.

The sclerotherapy catheter also has a second tube-formed or tube-like cavity 2, which has multiple outlet openings 4 before the balloon-like element 3. On the other end of the tube-formed cavity 2 there is an opening 8 through which a sclerosant to be applied can be fed in using a syringe-like system, which can then flow into a blood vessel inner wall through the outlet openings 4.

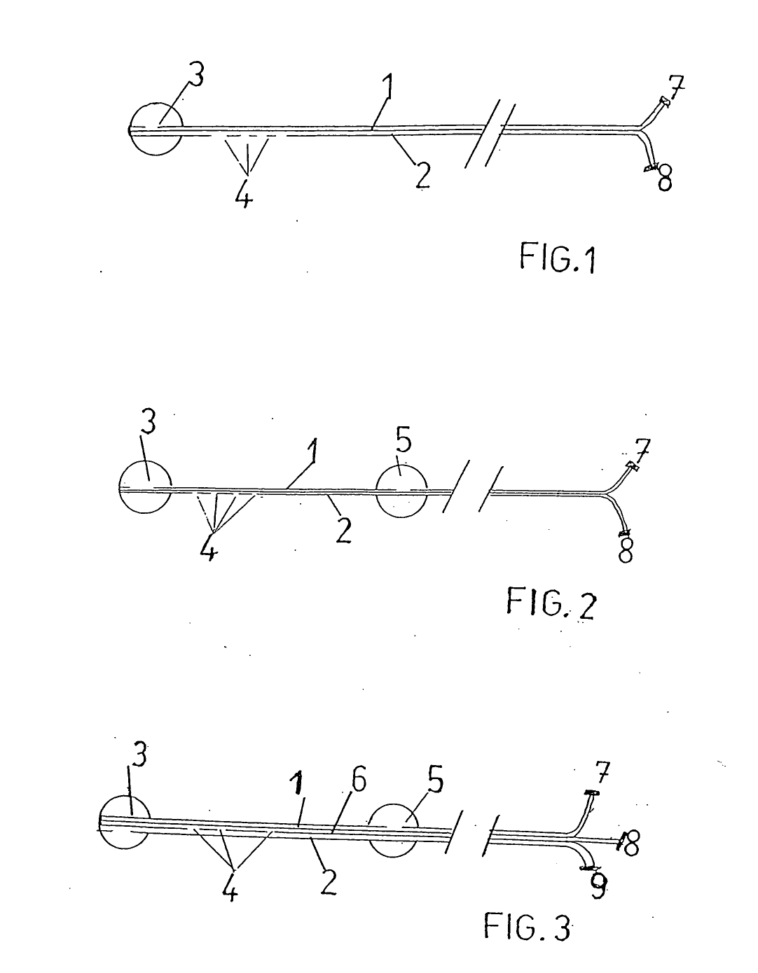

FIG. 2 shows a schematic representation of a second embodiment of the inventive sclerotherapy catheter.

In contrast to the sclerotherapy catheter shown in FIG. 1, this has a second balloon-like element 5 which communicates with the tube-formed cavity 1 through an interior opening.

FIG. 3 shows a schematic of a third embodiment of the inventive sclerotherapy catheter.

This sclerotherapy catheter has three tube-shaped cavities 1, 2, 6. Cavity 1 communicates over an interior opening with the balloon-like element 5, while cavity 2 communicates over an interior opening with the balloon-like element 3. The third cavity 6 has multiple outlet openings 4 between the two balloon-like elements 3, 5. All three cavities 1, 2, 6 can be filled with air or a sclerosant independently of one another (cavities 1 and 2 with air, cavity 6 with sclerosant.) At the ends of cavities 1, 2, 6 there are opening 7, 9, 8, through which a syringe-like system can be used to introduce air to be applied (through openings 7 and 9) or a sclerosant to be applied (through opening 8). The advantage of this somewhat more difficult construction of this catheter compared to the two previous embodiments is the fact that the two balloon-like elements 3 and 5 can be inflated independently of one another. This enables a fine adjustment to the physiological variables present in difficult applications.

To enable the insertion of the sclerotherapy catheter into curved blood vessels, the sclerotherapy catheter is constructed flexibly. In this way, the sclerotherapy catheter can conform itself during insertion into the blood vessel within certain limits to the path of the blood vessel, so that insertion into the blood vessel is eased. In the invention, the second cavity is constructed sufficiently rigidly along most of its overall length so that when an underpressure is generated in the second cavity to evacuate excess sclerosant from the vein, the closure of the second cavity due to contact of the second cavity's inner walls is prevented.

In the invention, the outlet openings 4 are in a fixed location relative to the first balloon-like element 3 and built into the tube-formed or tube-shaped cavity 2. Since the outlet openings 4 are built into the tube-formed cavity 2, which is constructed flexibly but still relatively rigidly, it is possible with the inventive catheter to evacuate excess sclerosant from the blood vessel after conclusion of the treatment. In this way it is prevented that excess sclerosant remains in the blood vessel. The evacuation of the sclerosant can, for example, be achieved by retracting the plunger of the syringe-like system, so that air is withdrawn from cavity 2, and the sclerosant is sucked into cavity 2.

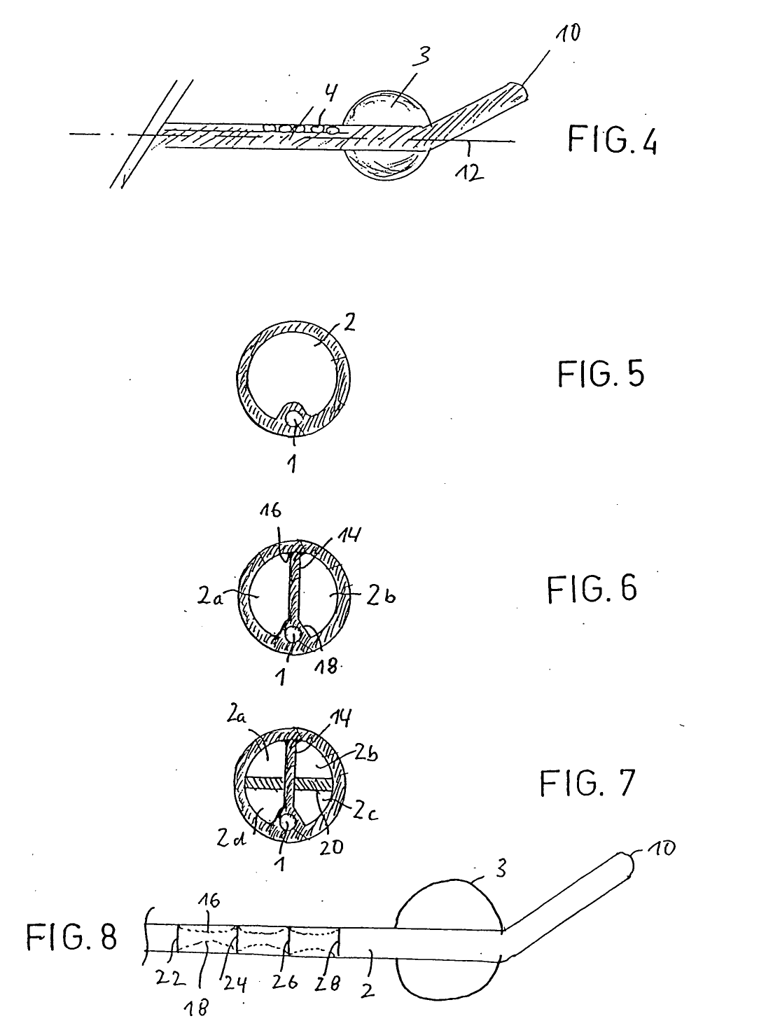

FIG. 4 shows a schematic of a fourth embodiment of the inventive sclerotherapy catheter, which differs from the embodiment shown in FIG. 1 in that a distal end 10 of the sclerotherapy catheter, referred to in brief as the catheter in the following, is bent by an angle of about 30° relative to a broken line 12 in FIG. 4 symbolizing a lengthwise axis of the catheter. The distal end 10 of the catheter can thereby be rounded in order to prevent injury to the interior wall of the blood vessel. The embodiment shown in FIG. 4 has the advantage that insertion of the catheter into the blood vessel is simplified. Should the distal end 10 of the catheter encounter an obstacle in the blood vessel, the catheter can be rotated around its lengthwise axis 12 into an orientation in which the distal end 10 no longer encounters the obstacle. For example, the catheter can be rotated such that the angle at the distal end 10 follows the path of the blood vessel.

The angle at which the distal end 10 of the catheter is bent can be chosen within a wide range.

Corresponding to user and treatment requirements, the distal end 10 of the catheter can also be constructed in a curved form.

FIG. 5 shows a cross-section of a fifth embodiment of the inventive catheter, in which the second cavity has a significantly greater cross-section than the first cavity 1. The first cavity 1 and the second cavity 2 are constructed in one piece as a tube section. Due to the one-piece construction of cavities 1, 2, the rigidity of the second cavity in the area where the first cavity 1 is located is increased.

FIG. 6 shows the cross-section of a sixth embodiment of the inventive catheter, which differs from the embodiment shown in FIG. 5 in that in order to increase the rigidity of the second cavity 2, stabilization structures are added, which in the embodiment shown in FIG. 6 take the form of a rib 14 which extends in the radial direction of the second cavity 2 between the opposing area 16, 18 of the interior wall of the second cavity. It is not obvious from the drawing, and thus explained here, that the rib 14 extends in the axial direction of the second cavity 2 over the entire length of the second cavity 2, so that the second cavity 2 is subdivided into two individual cavities 2a, 2b. The individual cavities 2a, 2b, depending on current requirements, can be separated from one another or communicate with one another. Rib 14 increases the rigidity of the second cavity 2, so that when an underpressure is applied to the second cavity 2 in order to evacuate excess sclerosant, any deformation of the second cavity in the radial direction is prevented or at least lessened in the direction of the rib.

By subdividing the second cavity 2 into two individual cavities 2a, 2b, a three-cavity catheter is created. Thus one of the individual cavities 2a, 2b may be used for example to evacuate the sclerosant, while the other individual cavity carries, for example, a saline solution which can be introduced into the blood vessel.

FIG. 7 shows a cross-section of a seventh embodiment of the inventive catheter, which differs from the embodiment shown in FIG. 6 in that in addition to the rib 14 there is another rib 20, also extending in the radial direction of the second cavity 2, but vertically to the first rib 14. Thanks to ribs 14, 20, upon generation of an underpressure in the second cavity 2, a contraction of the second cavity 2 in the direction of rib 14 as well as in the direction of rib 20 is prevented or at least lessened. In this way it can be particularly reliably prevented that the second cavity 2 can close upon generation of an underpressure due to contact of the interior walls 16, 18 of the second cavity 2. The second cavity 2 is subdivided into four individual cavities 2a, 2b, 2c, and 2d by ribs 14, 20, which, depending on the then existing requirements, in use, can communicate with one another or be separated from one another.

In FIG. 8, an eighth embodiment of the inventive catheter is shown, which differs from the embodiment shown in FIG. 6 in that instead of rib 14 extending in the axial direction of the second cavity 2 along the greater part of the length of the second cavity, there are a number of bars 22, 24, 26, 28 spaced along the axial direction of the second cavity. The bars 22, 24, 26, 28 can have the same cross-section as rib 14 shown in FIG. 6 or ribs 14, 16 shown in FIG. 7. They prevent the wall areas of the second cavity 2 from coming into contact upon generation of an underpressure in the second cavity 2. If an underpressure is generated in the second cavity 2, the second cavity 2 does not deform, or will deform only slightly, at the axial positions where bars 22, 24, 26, 28 are located. In axial areas lying between, the walls of the second cavity 2 deform elastically, where the material of the walls of the second cavity 2 and the material of the bars 22, 24, 26, 28 as well as the axial spacing of bars 22, 24, 25, 28 is chosen in such a way that at the largest possible underpressure the contact of opposing areas 16, 18 is prevented, as shown by dashed lines in FIG. 8.

Part of the invention is also the use of an inventive catheter in a process or method of sclerotherapy or sclerosing of blood vessels, particularly veins. Also a part of the invention is a process or method for sclerotherapy or sclerosing of blood vessels in which a catheter is inserted into the blood vessel, in which a sclerosant is introduced through the catheter into the blood vessel, and finally in which excess sclerosant is evacuated from the blood vessel, preferably through the first cavity of the catheter. Also a part of the invention is a process for sclerotherapy of blood vessels in which a catheter according to the invention, as described, is used.

While this invention has been described as having a preferred design, it is understood that it is capable of further modifications, and uses and/or adaptations of the invention and following in general the principle of the invention and including such departures from the present disclosure as come within the known or customary practice in the art to which the invention pertains, and as may be applied to the central features hereinbefore set forth, and fall within the scope of the invention or limits of the claims appended hereto.

1. Sclerotherapy catheter for sclerotherapy of blood vessels, comprising:
a) at least two cavities, a first cavity of the at least two cavities having a first balloon-like element which can be filled with air and inflated to block the blood vessel;
b) a second cavity of the at least two cavities having an interior wall and at least one outlet opening fixed in location relative to the first balloon-like element and configured for the application of a sclerosant; and
c) the second cavity being sufficiently rigid along its entire length, so that, in use, upon generation of an underpressure required for the evacuation of excess sclerosant from a blood vessel into the second cavity, closure of the second cavity by elastic deformation of the interior wall of the second cavity is prevented.

2. Sclerotherapy catheter as in claim 1, wherein:
a) the first cavity has a second balloon-like element offset relative to the at least one outlet opening, and the second balloon-like element can be filled with air and inflated, for further closing of a blood vessel, in use; and
b) the at least one outlet opening is located between the first and second balloon-like elements.

3. Sclerotherapy catheter as in claim 1, wherein:
a) a third cavity is provided that has a second balloon-like element offset relative to the at least one outlet opening, and the second balloon-like element can be filled with air and inflated, in use, for further closing of a blood vessel; and
b) the at least one outlet opening is located between the first and second balloon-like elements.

4. Sclerotherapy catheter as in claim 3, wherein:
a) at least one of the first and third cavities has an air blockage system for blocking air escaping from the at least one cavity, in use.

5. Sclerotherapy catheter as in claim 4, wherein:
a) the air blockage system includes one of a valve, a rubber plug, and a compression clamp.

6. Sclerotherapy catheter as in claim 1, wherein:
a) a distal end of the sclerotherapy catheter is one of curved and bent at an angle of less than 45° relative to a lengthwise axis of the sclerotherapy catheter.

7. Sclerotherapy catheter as in claim 3, wherein:
a) a wall of at least one of the first cavity, and the second cavity, and the third cavity includes an elastically deformable material.

8. Sclerotherapy catheter as in claim 1, wherein:
a) a stabilizing structure is provided for stiffening the second cavity.

9. Sclerotherapy catheter as in claim 8, wherein:
a) the stabilizing structure includes at least one of a rib extending in a radial direction between an interior wall of the second cavity and a bar extending in a radial direction between the interior walls of the second cavity.

10. Sclerotherapy catheter as in claim 9, wherein:
a) the at least one of the rib and the bar extends in an axial direction largely over an entire length of the second cavity.

11. Sclerotherapy catheter as in claim 9,
a) the at least one of the rib and bar includes at least one of multiple ribs and bars, and each of which extend over a portion of a length of the second cavity and are spaced apart from one another along an axial direction.

12. Sclerotherapy catheter as in claim 10, wherein:
a) the second cavity is subdivided by the at least one of the rib and the bar into at least two individual cavities.

13. Sclerotherapy catheter as in claim 12, wherein:
a) the at least two individual cavities are separated from one another.

14. Sclerotherapy catheter as in claim 12, wherein:
a) the at least two individual cavities communicate with one another.

15. Process for sclerotherapy of blood vessels, comprising the steps of:
a) providing a sclerotherapy catheter, the sclerotherapy catheter including:
i) a first cavity having a first balloon-like element which can be filled with air and inflated to block the blood vessel;
ii) a second cavity having an interior wall and at least one outlet opening fixed relative to the first balloon-like element and the at least one outlet opening being configured for the application of a sclerosant; and
iii) the second cavity being sufficiently rigid along its entire length, so that, in use, upon generation of an underpressure required for the evacuation of excess sclerosant from a blood vessel into the second cavity, closure of the second cavity by elastic deformation of the interior wall of the second cavity is prevented;
b) inserting the catheter into a blood vessel; and
c) introducing a sclerosant into the blood in the blood vessel through the sclerotherapy catheter.

16. Process for sclerotherapy of blood vessels as in claim 15, wherein:
a) the first cavity of the sclerotherapy catheter has a second balloon-like element offset relative to the at least one outlet opening, and the second balloon-like element can be filled with air and inflated, for further closing of a blood vessel, in use; and
b) the at least one outlet opening is located between the first and second balloon-like elements.

17. Process for sclerotherapy of blood vessels, comprising:
a) inserting a sclerotherapy catheter into a blood vessel;
b) introducing a sclerosant into blood in the blood vessel through the sclerotherapy catheter; and,
c) after the step of introducing the sclerosant into the blood in the blood vessel, then excess sclerosant is evacuated from the blood vessel.

18. Process as in claim 17, wherein:
a) in the step of evacuating excess sclerosant from the blood vessel the catheter of claim 1 is used to evacuate the excess sclerosant.

19. Process as in claim 17, wherein:
a) the sclerotherapy catheter includes a first cavity; and
b) the excess sclerosant is evacuated through the first cavity of the sclerotherapy catheter.

20. Process as in claim 19, wherein:
a) the blood vessel is a vein.

21. Process as in claim 17, wherein:
a) the blood vessel is a vein.
